(19) United States
(12) Patent Application Publication
(10) Pub. No.: US 2012/0142550 A1
(43) Pub. Date: Jun. 7, 2012

(54) Vanin 1 as a Peripheral Blood Oxidative Stress Sensor

(76) Inventors: James L. Zehnder, Menlo Park, CA (US); Bing Zhang, Fremont, CA (US)

(21) Appl. No.: 13/311,367

(22) Filed: Dec. 5, 2011

Related U.S. Application Data
(60) Provisional application No. 61/420,137, filed on Dec. 6, 2010.

(51) Int. Cl. C40B 30/04; C40B 30/00; G01N 33/566; C12Q 1/68

(52) U.S. Cl. 506/9; 435/6.11; 506/7; 435/6.12; 435/7.1; 435/7.92

(57) ABSTRACT

Aspects of the subject invention are drawn to methods, compositions, systems and kits for the assessment of oxidative stress in an individual from a blood sample. In certain embodiments, the expression level of VNN1 in blood cells from a subject (or patient) is assessed and used to determine the subject's oxidative stress state, where an increased/high expression level of VNN1 indicates that the subject is in a state of oxidative stress. Expression of VNN1, and optionally other genes, may be done by assessing nucleic acid and/or protein levels in the blood cells obtained from the subject.

### INTRODUCTION

Oxidative stress in an individual is implicated in pathogenesis and progression of many diseases, including infectious, inflammatory, autoimmune, and cardiovascular diseases. Methods for determining whether a subject is in a state of oxidative stress thus find use in the clinic, e.g., for diagnostic, prognostic, risk analysis and therapeutic intervention applications.

### SUMMARY

Aspects of the subject invention are drawn to methods, compositions, systems and kits for the assessment of oxidative stress in an individual from a blood sample. In certain embodiments, the expression level of VNN1 in blood cells from a subject (or patient) is assessed and used to determine the subject's oxidative stress state, where an increased/high expression level of VNN1 indicates that the subject is in a state of oxidative stress. Expression of VNN1, and optionally other genes, may be done by assessing nucleic acid and/or protein levels in the blood cells obtained from the subject.

Certain aspects of the subject invention are drawn to methods of determining whether a subject is experiencing oxidative stress by evaluating the level of expression of a VNN1 expression product in cells of hematopoietic lineage, or blood cells, from the subject to obtain a gene expression result and then determining whether the subject is experiencing oxidative stress based on this result gene expression result, where an elevated level of a VNN1 expression product in the blood cells indicates that the subject is experiencing oxidative stress. The VNN1 expression product can be a nucleic acid transcript or protein. Additional genes may also be evaluated, e.g., PPARγ (where a reduced level a PPARγ expression product in the blood cells further indicates that the subject is experiencing oxidative stress).

Additional aspects of the subject invention are drawn to methods of managing treatment of a subject having a disease condition, e.g., ITP, by determining whether the subject is experiencing oxidative stress (as described above) and then managing treatment of the subject based on the determination.

Additional aspects of the subject invention are drawn to systems and kits for determining whether a subject is experiencing oxidative stress which include a gene expression evaluation element for evaluating the level of expression of a VNN1 expression product in blood cells from the subject to obtain a gene expression result and an oxidative stress determination element for employing the gene expression result to determine whether the subject is experiencing oxidative stress.

Computer program products for determining whether a subject is experiencing oxidative stress are also provided, where the computer program product, when loaded onto a computer, is configured to employ a gene expression result from blood cells derived from the subject to determine whether the subject is experiencing oxidative stress, and where the gene expression result includes expression data for VNN1.

### BRIEF DESCRIPTION OF THE DRAWINGS

FIG. 1. Unsupervised hierarchical clustering pattern of expression data. Each row represents a single transcript and each column represents a single sample. Red indicates greater expression, green indicates lower expression, and grey indicates missing data. In the sample dendrogram, self-limited acute ITP samples are in red, chronic ITP samples are in blue, and normal controls are in green. (A) Unsupervised clustering of self-limited acute and chronic ITP patients using the transcripts with significantly elevated expression in chronic ITP at the SAM q-value of 0 (using clones corresponding to putative genes and >80% good data, 57 biosequences passed the filters). Two distinct clusters of samples are revealed: the one on the left contains predominantly chronic ITP samples while the one on the right contains only self-limited acute ITP samples. (B) Unsupervised clustering of self-limited acute ITP, chronic ITP and healthy controls using the same set of transcripts and the same filters settings (57 biosequences passed filters). The expression level of these transcripts presented a low-to-high gradual transition from normal to chronic ITP. While positioned in the middle, the expression pattern of self-limited acute ITP has greater similarity to that of healthy controls.

FIG. 2. Pathway analysis by IPA®. (A) Significantly altered canonical pathways associated with chronic ITP in comparison to self-limited acute ITP. A total of 535 transcripts had a q-value <5% by SAM analysis. These transcripts were mapped to 338gene IDs in the Ingenuity® Pathway Analysis database and then analyzed by the IPA® software to identify the most significantly perturbed canonical pathways. The canonical pathways included in this analysis are displayed along the x-axis of the bar chart. The y-axis displays the statistical significance on the left and the ratio on the right. Calculated using the right-tailed Fisher Exact Test, the p-value indicates which biological annotations are significantly associated with the input molecules relative to all functionally-characterized mammalian molecules. The yellow threshold line represents the default significance cutoff at p=0.05. The ratio was calculated by taking the number of genes from the dataset that participate in a canonical pathway, and dividing it by the total number of genes in that canonical pathway. The ratio indicates the percentage of genes in a pathway that were also found in the uploaded genes. (B) Significantly altered toxicity lists associated with chronic ITP. These lists have been grouped based on critical biological processes and toxicological responses. Only 5 toxicology lists reached statistical significance: PPAR, NFκB, and oxidative stress pathways are predominant.

FIG. 3. (A) Real-time PCR validation of VNN1 expression in different ITP groups and healthy controls. Five groups of samples were included in the validation: Self-limited acute ITP (A, n=8), Acute-to-chronic ITP (A-C, n=7), Healthy control (N, n=5), Resolved acute ITP (A-R, n=6), and Chronic ITP resistant to multiple treatments (RC, n=6). The non-parametric Mann-Whitney two-tailed test was performed in the statistical analysis. At the transcriptional level, VNN1 expression in the A-C group is significantly higher compared to the A (p=0.0093), N (p=0.0177) and A-R (p=0.0221) groups; VNN1 expression in the RC group is significantly higher than the A group (p=0.0127). The upper and lower limits of each box represent the 75th and 25th percentiles, respectively; the horizontal lines inside the box represent medians; the whiskers represent extreme measurements. (B) Expression distribution of VNN1 in subsets of human blood cells. Purified CD15+ granulocytes, CD20+ B cells, CD14+ monocytes, CD3+ CD4+ T cells, CD3+ CD8+ T cells and platelets were obtained from blood donors as described in Methods. The relative expression level of VNN1 determined by real-time PCR in normal human adults is high in granulocytes and monocytes and moderate in platelets. VNN1 expression is low in CD4+ T cells, CD8+ T cells and B cells. The range and mean of normalized VNN1 expression value in each cell subset are shown.

FIG. 4. Expression changes of VNN1 and PPARγ in response to oxidative stress inducers. PBMC samples were treated with LPS or sodium arsenite and cultured for 12 hours, after which the cells were harvested and VNN1 and PPARγ expression were measured by real-time PCR. The expression fold changes were calculated by dividing the normalized VNN1 and PPARγ expression values in treated cells by values in non-treated cells. (A) After LPS treatment, VNN1 increased 5˜40 fold while PPARγ decreased 25˜76 fold. (B) After sodium arsenite treatment, VNN1 increased 2˜40 fold while PPARγ decreased 4˜7 fold.

FIG. 5. GSH/GSSG ratio in ITP patients and controls. The concentrations of GSH and GSSG in whole blood were measured in ITP patients and pediatric healthy controls; thereafter, the GSH/GSSG ratio was calculated for each sample and unpaired t-test was performed to compare GSH/GSSG ratio between groups (presented as mean±SEM). (A) The whole blood GSH/GSSG ratio is significantly lower in ITP patients in general (p=0.0011), indicating a higher oxidative stress state compared to healthy controls. The GSH/GSSG ratio is also significantly lower in chronic ITP patients (p=0.0154) compared to healthy controls. (B) The GSH/GSSG is significantly higher in patients with recent treatments (within 1 month of sample collection) as compared to patients without recent treatments (p=0.0035).

FIG. 6. Schematic representation of the postulated Vanin-1 pathway in human blood cells in response to oxidative stress. This figure summarizes our hypothesis based on Berruyer et al's work (J Exp Med. 2006; 203(13):2817-27; and Mol Cell Biol. 2004; 24(16):7214-24) and our findings. The steps with experiment data support are highlighted in box. An inciting event (e.g., infection) induces generation of free radical species, while ROS has a positive modulatory role in immune activation and eradication of viral infections, excessive ROS or inadequate capability of antioxidant scavengers leads to an oxidative stress state. In the presence of oxidative stress, antioxidant response-like elements within the promoter region of VNN1 act as stress-regulated targets and enhance VNN1 expression. More cysteamine is produced from hydrolysis of pantethine; cysteamine is then converted to cystamine, which is an inhibitor of gamma-glutamylcysteine synthetase (γ-GCS), the rate-limiting enzyme of glutathione synthesis. Thus the glutathione store as well as the GSH/GSSG ratio decrease, which subsequently intensifies the oxidative stress. On the other hand, the anti-inflammatory check-point PPARγ is also antagonized by cystamine and as a result, more inflammatory cytokines and chemokines are produced.

### DETAILED DESCRIPTION OF THE INVENTION

Aspects of the subject invention are drawn to methods, compositions, systems and kits for the assessment of oxidative stress in an individual from a blood sample. As described herein, an increased/high expression level of VNN1 in blood cells from a subject indicates that the subject is in a state of increased oxidative stress. Expression of VNN1, and optionally other genes, may be done by assessing nucleic acid and/or protein levels in the blood cells obtained from the subject.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Oxidative stress is implicated in pathogenesis and progression of many diseases—e.g., infectious, inflammatory, autoimmune, and cardiovascular. The ability to accurately assess oxidative stress levels has diagnostic utility. As detailed below, we have defined the range of expression of VNN1 on various subsets of peripheral blood cells and demonstrated dramatic up-regulation of this gene with oxidative stress inducers, such as low dose LPS and sodium arsenite. In studies of gene expression of ITP patients, we found that oxidative stress pathways in general and VNN1 in particular, are implicated in the pathogenesis of the chronic form of the disease.

### Methods

Aspects of the subject invention provide methods of determining whether a patient or subject is experiencing oxidative stress. By oxidative stress is meant any of various pathologic changes seen in living organisms, including humans, in response to excessive levels of cytotoxic oxidants and free radicals in the environment. Oxidative stress can be generally defined as an imbalance of the prooxidant/antioxidant ratio in which too few antioxidants are produced or ingested or too many oxidizing agents are produced. Thus, oxidative stress is a term used to describe the effect of oxidation in which an abnormal level of reactive oxygen species (ROS), such as free radicals (e.g., hydroxyl, nitric acid, superoxide) or non-radicals (e.g. hydrogen peroxide, lipid peroxide) lead to oxidative damage to specific molecules with consequential injury to cells or tissue. Increased production of ROS can occur in a variety of ways, including as a result of infection (e.g., fungal or viral), inflammation, ageing, UV radiation, pollution, excessive alcohol consumption, cigarette smoking, etc. As detailed herein, we have found that oxidative stress pathways are implicated in the pathogenesis of the chronic form of Immune Thrombocytopenia (ITP).

In practicing the subject methods, blood cells from a subject (or patient) are assayed to determine the level of expression of Vanin-1 (VNN1) to determine whether the subject is experiencing oxidative stress. VNN1 is a GPI anchored ectoenzyme with pantetheinase activity. VNN1 is an indirect inhibitor of PPARG and pro-inflammatory function in epithelial cells. (Entrez Gene ID: 8876). As demonstrated in the Experimental section below, an increased expression level of VNN1 in blood cells of a subject is associated with oxidative stress in the subject (which can be caused by any of a variety of conditions, as noted above).

In practicing the subject methods, blood cells are obtained from the subject or patient of interest, also referred to herein as a blood sample or blood-derived sample. In many embodiments the sample is derived from blood cells harvested from whole blood. Of particular interest as a sample source is peripheral blood. Any convenient protocol for obtaining such samples may be employed, where suitable protocols are well known in the art. The blood sample may be subjected to a processing step prior to analysis. For example, the blood sample may be subjected to processes in which specific cells or cell subsets are enriched, e.g., lymphocytes, monocytes, granulocytes, etc. Enrichment may be carries out in any convenient manner, including magnetic activated cell sorting (e.g., using Miltenyi MACS® Cell Separation), flow cytometry, gradient-density centrifugation, differential cellular lysis protocols, etc.

In practicing the subject methods, the blood cells in the sample are assayed to determine the expression level of VNN1 (and optionally other genes). In other words, an expression level value for VNN1 in the cells in the sample is obtained. “Expression level” is used broadly to include an expression level of nucleic acid transcripts, e.g., mRNAs, or a proteomic expression level, e.g., an expression level of VNN1 protein. Exemplary assays for determining the expression level of VNN1 are provided below.

In certain embodiments, the cells in the sample are assayed for the expression level of additional genes, e.g., two or more, e.g., 5 or more, 10 or more, 15 or more, 25 or more, 50 or more, 100 or more, 200 or more, etc., genes may be evaluated. In certain embodiments, one of the additional genes evaluated is PPARγ. As detailed below, PPARγ expression is downregulated in blood cells during oxidative stress. In certain embodiments, the evaluation that is made may be viewed as an evaluation of the transcriptome, as that term is employed in the art. See e.g., Gomes et al., Blood (2001 Jul. 1) 98(1):93-9. Thus, in many embodiments, a sample is assayed to generate an expression profile (or signature) that includes expression data for VNN1 and at least one additional gene/protein, and sometimes a plurality of genes/proteins, where by plurality is meant at least two additional genes/proteins, and often at least 5, at least 10, at least 20 different genes/proteins or more, such as 50 or more, 100 or more, etc.

In the broadest sense, the expression level obtained, or determined, in the subject methods may be qualitative or quantitative. As such, where detection is qualitative, the methods provide a reading or evaluation, e.g., assessment, of whether or not the target analyte, e.g., nucleic acid or protein, is present in the sample being assayed. In yet other embodiments, the methods provide a quantitative detection of whether the target analyte is present in the sample being assayed, i.e., an evaluation or assessment of the actual amount or relative abundance of the target analyte, e.g., nucleic acid or protein in the sample being assayed. In such embodiments, the quantitative detection may be absolute or relative, e.g., relative to another analyte in the same blood sample or to a separate blood sample, e.g., a positive or negative control sample. As such, the term “quantifying” when used in the context of quantifying a target analyte in a sample can refer to absolute or to relative quantification. Absolute quantification may be accomplished by inclusion of known concentration(s) of one or more control analytes and referencing the detected level of the target analyte with the known control analytes (e.g., through generation of a standard curve).

In certain embodiments, the expression level of VNN1, and optionally other genes, is determined by detecting the amount or level of VNN1 gene-derived nucleic acids in the sample, e.g., nucleic acid transcripts. In certain of these embodiments, nucleic acids are obtained from the cells in the blood sample to produce a nucleic acid sample. The nucleic acids in the nucleic acid sample may include RNA or DNA, e.g., mRNA, cRNA, cDNA etc., so long as the sample retains the expression information of the blood cells from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from blood cells harvested from a subject using standard protocols, including, but not limited to, peripheral blood cells, etc., as reviewed above.

The expression levels (or expression profile) of the nucleic acid analytes of interest in the cells of the blood sample, including VNN1 and optionally PPARγ, may be determined from the nucleic acid sample using any convenient protocol. Non-limiting examples include hybridization detection based assays, e.g., Northern blots, microarray analysis, etc., as well as amplification based assays (e.g., linear or non-linear amplification methods), including those that employ the Polymerase Chain Reaction (PCR), e.g., quantitative PCR, reverse-transcription PCR (RT-PCR), real-time PCR, and the like.

A variety of different manners of determining expression levels (or generating expression profiles) are known, such as those employed in the field of differential gene expression analysis. One representative and convenient type of protocol for generating expression profiles is array-based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions. “Stringent hybridization conditions” and “stringent hybridization wash conditions in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO4, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate. The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptome), may be both qualitative and quantitative.

Where the expression level determination of VNN1, and optionally other genes, is a protein expression level determination, any convenient protein detection protocol may be employed. Representative methods include, but are not limited to: proteomic arrays (e.g., arrays of analyte specific antibodies or binding fragments), flow cytometry, standard immunoassays (e.g., western blot, ELISA assays, immunohistochemistry), mass spectrometry, etc.

In many embodiments, the determination of the protein level of VNN1, and optionally other proteins, is done using flow cytometry. As used herein, the term “flow cytometry” refers to a method by which the individual cells of a sample are analyzed by their optical properties (e.g., light absorbance, light scattering and fluorescence properties, etc.) as they pass in a narrow stream in single file through a laser beam. Flow cytometry methods include fluorescence activated cell sorting (FACS) methods by which a population of cells having particular optical properties are separated from other cells.

In general methods of flow cytometry, cells in a blood sample are contacted with a detectable binding agent specific for the analyte of interest, e.g., VNN1, under conditions that allow for specific binding of the binding agent to its target analyte (protocols for staining cells with binding agents for flow cytometric analysis are well known in the art). A binding agent in many embodiments of the invention is an antibody, or antigen binding fragment thereof, that is specific for the analyte, e.g., VNN1. Antibodies may be derived from any number of sources, e.g., mouse, rat, rabbit, etc., and may be monoclonal or polyclonal, as is well known in the art. By “detectable” is meant that the binding agent can be detected, either directly or indirectly. For example, an antibody may be fluorescently labeled with a fluorophore or a quantum dot which can be detected by flow cytometry. After contacting the cells and the binding agent, the amount of antibody specifically binding to the cells (i.e., via interaction with its target analyte) is determined using flow cytometry, thereby obtaining an evaluation of VNN1 protein level in the cells from the population of blood cells. Flow cytometry methods are known and have been reviewed in a variety of publications, including Brown et al (Clin Chem. 2000 46:1221-9), McCoy et al (Hematol. Oncol. Clin. North Am. 2002 16:229-43) and Scheffold J. Clin. Immunol. 2000 20:400-7) and books such as Carey et al (Flow Cytometry in Clinical Diagnosis, 4^{th} Edition ASCD Press, 2007), Ormerod (Flow Cytometry—A practical approach 3rd Edition. Oxford University Press, Oxford, UK 2000), Ormerod (Flow Cytometry 2nd Edition. BIOS Scientific Publishers, Oxford, UK 1999) and Ormerod (Flow Cytometry—A basic introduction 2009 Cytometry Part A 75A, 2009), which are all incorporated by reference herein for disclosure of those methods.

In some embodiments, other analyte specific binding agents are included in the assay. For example, antibodies that recognize analytes that identify specific cell subsets may also be used and detected, e.g., antibodies for T cells like CD4 or CD8, antibodies for B cells like B220, etc. These additional binding agents when used are generally differentially labeled such that their detection can be differentiated from the binding element for VNN1 (such assays are also known in the art as multi-parameter flow cytometry).

The flow cytometry-based methodology described herein may be carried out on any suitable flow cytometer, examples of which are known on the art and described in, e.g., U.S. Pat. Nos. 5,378,633, 5,631,165, 6,524,858, 5,266,269, 5,017,497 and 6,549,876, PCT publication WO99/54494 and as well as published U.S. Patent Applications US20080153170, 20010006787, US20080158561, US20100151472, US20100099074, US20100009364, US20090269800, US20080241820, US20080182262, US20070196870 and US20080268494, each of which are incorporated by reference herein).

The data obtained for the expression level of VNN1 (and optionally other genes) in the blood sample from the subject is used to determine whether the subject is undergoing oxidative stress. In certain embodiments, the expression level for VNN1 obtained is compared with a reference or control level for VNN1 to determine whether the subject is undergoing oxidative stress. The terms “reference” and “control” as used herein mean a standardized of gene expression levels for VNN1 (and optionally other genes of interest, e.g., PPARγ) used to interpret the expression level value of the cells in a blood sample from a subject, thus allowing the user of the subject methods to determine the oxidative stress status of the subject. The reference or control levels may be obtained from blood cells of a subject known to have a desired phenotype, e.g., having oxidative stress, and therefore may be a positive reference or control. In addition, the reference/control may be from blood cells of a subject known to not have the desired phenotype, e.g., not having oxidative stress, and therefore be a negative reference/control. As noted above, an increase in the expression level of VNN1, e.g., over that of a negative control, indicates that the subject is undergoing oxidative stress.

In certain embodiments, the obtained expression level data is compared to a single reference/control to obtain information regarding the phenotype of the subject whereas in other embodiments, the obtained expression level data is compared to two or more different references/controls to obtain more in depth information regarding the phenotype of the subject. For example, the obtained expression level data may be compared to a positive and negative control to obtain confirmed information regarding whether the subject is undergoing oxidative stress.

The comparison of the obtained expression levels (or profile) and the one or more references/controls may be performed using any convenient methodology, where a variety of methodologies are known to those of skill in the array art, e.g., by comparing digital images or representations of the expression levels, by comparing databases of expression level data, etc. Patents describing ways of comparing expression levels/profiles include, but are not limited to, U.S. Pat. Nos. 6,308,170 and 6,228,575, the disclosures of which are herein incorporated by reference. The comparison step results in information regarding how similar or dissimilar the obtained expression level is to the control/reference, which similarity/dissimilarity information is employed to determine the phenotype of the subject. For example, similarity with a positive control indicates that the subject is undergoing oxidative stress. Likewise, similarity with a negative control indicates that the subject is not undergoing oxidative stress.

In certain embodiments, the gene oxidative stress result obtained by the subject methods is compared to other methods for oxidative stress assessment in a subject, a number of which are known in the art. For example, the oxidative stress result may be compared to an assessment of glutathione (GSH, reduced form) and glutathione disulfide (GSSG, oxidized form) from the same subject (e.g., as describe in the Experimental section below).

The subject methods further find use in pharmacogenomic applications. In these applications, a subject/host/patient is first diagnosed for the presence or absence of oxidative stress as described in the preceding section. The subject is then treated using a protocol whose suitability is determined using the results of the diagnosis step. More specifically, where the subject is identified as undergoing oxidative stress, a protocol that may include counteracting the oxidative stress and/or the underlying condition leading to the oxidative stress may be employed to manage/treat the subject. In certain embodiments where a subject or patient is identified as not undergoing oxidative stress, certain treatments may be counter-indicated. Such an evaluation can thus be used to prevent unnecessary treatments of a patient.

In many embodiments, a subject is screened for the presence of oxidative stress following or during treatment of a disease or condition (e.g., cancer treatment, transplantation of an organ, treatment of an infection, treatment of an autoimmune disease, etc.). The subject or patient may be screened once or serially following such treatments, e.g., weekly, monthly, bimonthly, half-yearly, yearly, etc. In certain embodiments, monitoring of the host blood cell expression level of VNN1, and optionally other genes, is conducted to determine whether the treatment was curative or temporary.

### Systems and Kits

Also provided are systems and kits for practicing one or more of the above-described methods. The subject systems and kits may vary greatly, but typically include at least an gene expression evaluation element, e.g., one or more reagents, and a phenotype determination element.

Reagents of interest include reagents specifically designed for use in generating expression profiles for VNN1, and optionally other genes i.e., a gene expression evaluation element. One type of such reagent is a probe nucleic acid (e.g., on a microarray or in solution) for the genes of interest. Where the gene expression evaluation element is a microarray, a variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

Another type of reagent that is specifically tailored for generating expression profiles of VNN1 and optionally other genes of interest is a collection of gene specific primers designed to selectively amplify such genes. Gene specific primers and methods for using the same are described in U.S. Pat. No. 5,994,076, the disclosure of which is herein incorporated by reference. Of particular interest are collections of gene specific primers that have primers for VNN1 and optionally other genes, e.g., PPARγ.

The systems and kits of the subject invention may include the above-described probes, arrays and/or gene specific primer collections. The systems may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

Another type of reagent that is specifically tailored for determining the expression level of VNN1 in blood cells (and optionally other genes of interest) is a detectable binding agent specific for VNN1. Examples include detectably labeled antibodies or VNN1 binding fragments thereof either in solution or immobilized to a substrate, e.g., a plate, bead, microarray, etc., as is known in the art. Such reagents may be used in flow cytometry, western blots, ELISAs, etc., to determine the expression level of VNN1 in blood cells, or a sample derived therefrom.

The systems and kits may also include a phenotype determination element, which element is, in many embodiments, a reference or control expression profile that can be employed, e.g., by a suitable computing means, to make a phenotype determination based on an “input” expression level, e.g., that has been determined with the above described gene/protein expression evaluation element. Representative phenotype determination elements include databases of expression profiles, e.g., reference or control profiles, as described above.

The subject systems and kits may also include one or more other reagents for preparing or processing polynucleotides according to the subject methods. The reagents may include one or more matrices, solvents, sample preparation reagents, buffers, desalting reagents, enzymatic reagents, denaturing reagents, where calibration standards such as positive and negative controls may be provided as well. As such, the kits may include one or more containers such as vials or bottles, with each container containing a separate component for carrying out a sample processing or preparing step and/or for carrying out one or more steps for producing a normalized sample according to the present invention.

In addition to above-mentioned components, the subject kits typically further include instructions for using the components of the kit to practice the subject methods, e.g., to prepare blood samples and determine the expression level of VNN1 and optionally other genes in the blood sample. The instructions for practicing the subject methods are generally recorded on a suitable recording medium that can be read/accessed by a user of the system/kit. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control samples and reagents, e.g., two or more control samples for use in testing the kit.

### Exemplary Utility

In some applications the methods, systems and kits of the subject application may be employed as a prognostic marker of disease progression in ITP patients. As detailed herein, determining the level of expression of Vanin-1 transcript or protein can be used to predict disease progression in ITP patients at an early stage so that more appropriately tailored treatment regimens can be administered to prevent the patient from developing chronic disease. Moreover, because VNN1 is associated generally with oxidative stress in a subject, it represents a novel target molecule whose expression can be evaluated to identify oxidative stress in other disease/pre-disease states. Thus, the methods, systems and kits described herein find use in evaluating other autoimmune disease states in which oxidative stress is considered a causative factor (e.g., systemic lupus erythematosus (SLE), type 1 diabetes mellitus (T1DM), rheumatoid arthritis (RA), and systemic sclerosis (SS), autoimmune hemolytic anemia (AIHA), etc.)

The findings described herein also suggest VNN1 as novel therapeutic target for redox and inflammation modulation. Blocking Vanin-1 expression has been associated with increased resistance to oxidative stress and decreased inflammatory reactions. Therefore, reducing the lipid/protein peroxidation and the consequent neoantigen formation, may prevent disease progression. In addition, cancer stem cells have been shown to have increased GSH levels, which may accounts for the resistance to radiation. As such, ways to abrogate GSH are now actively sought.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Immune thrombocytopenia (ITP) is an immune-mediated hematological disorder in which increased platelet destruction and decreased platelet production lead to thrombocytopenia and mucocutaneous bleeding. The pathophysiology of ITP has been extensively investigated. It is generally accepted that a complex multi factorial immune dysregulation, loss of immune tolerance, and generation of platelet autoantibodies account for the primary mechanism. Nevertheless, the underlying pathogenic events leading to the breakdown of immune tolerance in ITP remain elusive. Molecular mimicry and epitope spreading theories provide plausible explanations for the appearance of autoantibodies; however, most patients have antibodies against multiple platelet surface glycoproteins at the time the disease becomes clinically evident, and the factors that initiate autoantibody production, as well as the reason for derivation of cryptic epitopes in vivo are still unknown. Protein modification as a result of free radical-mediated oxidative damage has been shown to elicit antibodies in a number of autoimmune diseases, including systemic lupus erythematosus (SLE), type 1 diabetes mellitus (T1DM), rheumatoid arthritis (RA), and systemic sclerosis (SS). (Kurien B T, et el., Free Radic Biol Med. 2006; 41 (4):549-56). More recently, evidence from a murine model also confirmed the role of reactive oxygen species (ROS) in triggering the autoimmune reaction in autoimmune hemolytic anemia (AIHA). (Luchi Y et al. Free Radic Biol Med. 2010; 48(7):935-44.) Griffiths (Autoimmun Rev. 2008; 7(7):544-9) points out in his review that while low levels of oxidants are important as signaling molecules, oxidant over-production in the absence of adequate antioxidant defense may cause irreversible changes to biomolecules and contribute to disease progression; generation of antigenic determinants by ROS and reactive nitrogen species (RNS) may contribute to epitope spreading in autoimmunity.

In children, ITP is typically preceded by a viral illness, and while the majority of patients resolve spontaneously within 6 months of diagnosis, about 20% of patients develop chronic disease. The underlying mechanism distinguishing self-limited acute ITP from chronic ITP is unknown. Knowledge of these differences would not only contribute to a better understanding of the pathogenesis of ITP, but also identify potential targets for therapeutic interventions in the group at risk for chronic ITP.

In the present study, we used whole transcriptome cDNA microarray analysis of peripheral blood as a tool to analyze the gene expression profiles of acute and chronic ITP patients. Oxidative stress-related pathways were revealed to be among the most significantly perturbed canonical pathways in chronic ITP and this was a distinguishing feature of chronic versus acute disease. Of particular interest was the increased expression of the gene Vanin-1 (VNN1) in progression-to-chronic ITP patients and treatment-resistant chronic ITP patients. VNN1 is known to play a role in oxidative stress response and license the production of inflammatory mediators in intestinal epithelial cells by antagonizing peroxisome proliferator-activated receptor gamma (PPARγ). (Berruyer C et al., J Exp Med. 2006; 203(13):2817-27) PPARγ is known to be an anti-inflammatory check-point in many inflammatory settings and various cell types. (Szanto A, et al. Immunobiology. 2008; 213(9-10): 789-803.)

We demonstrate the expression distribution of VNN1 in the major subsets of human blood cells, and furthermore, a similar role of VNN1 as an oxidative stress sensor in human peripheral blood mononuclear cells (PBMCs). Thus, VNN1 in particular and oxidative stress pathways in general appear to be associated with the development of chronic ITP in children.

### Material and Methods

### Patient Enrollment and Blood Specimen Collection.

Subjects consented and enrolled into the study included pediatric patients (age <18 year) diagnosed as having primary immune thrombocytopenia (platelet count <150,000/μL), as well as pediatric controls (normal platelet count and no concurrent illnesses or medications at the time of blood draw). For microarray analysis and real-time PCR validation, 2.5 ml whole blood samples (8 samples from acute ITP patients with active disease, 14 samples from chronic ITP patients with active disease after 6 months of diagnosis, 7 samples from chronic ITP patients with active disease within 6 months of diagnosis, 6 samples from resolved acute ITP patients, and 5 samples from healthy pediatric controls) were collected into PAXgene™ Blood RNA tubes (PreAnalytiX, Hombrechtikon, Switzerland). Acute ITP patients were followed for at least 6 months to determine clinical outcomes. For glutathione measurement, whole blood specimens from 5 acute ITP patients with active disease, 4 chronic ITP patients within 6 months of diagnosis, 2 ITP patients within 6 months of diagnosis with outcome status pending, 16 chronic ITP patients with active disease after 6 months of diagnosis, and 15 pediatric healthy controls were collected in EDTA anticoagulant tubes and kept on ice. Table 1 shows the clinical characteristics of patients in the study. The study was approved by the Stanford University Institutional Review Board and consent forms were obtained from all patients and controls.

TABLE 1
Demographic and clinical charateristics of ITP patients.

| | | | | | | Platelet | | Recent | |
|---|---|---|---|---|---|---|---|---|---|
| | | | | | | count | | Treatments | |
| | | | | | | at time | | (within | |
| | | | | | | of blood | | 1 month of | |
| | Categories of ITP samples | # of | Sample | | Age | draw | Time from | sample | Additional |
| Experiments | based on disease pregression | samples | ID | Sex | (years) | (×10 \hat{ } g/L) | diagnosis | collection) | information |
| Microarray | Self-limited acute ITP | n = 8 | 1* | M | 4 | 2 | 1 day | MG | |
| and/or | (Blood was collected within 6 | | 2* | M | 4 | 98 | 3 days | | |
| realtime-PCR | months of diagnosis when the | | 3* | F | 14 | 39 | 1 week | | |
| | patients had active disease; | | 4* | M | 5 | 44 | 1 month | | |
| | patients resolved within 6 | | 5* | M | 1 | 35 | 1 month | | |
| | months) | | 6* | M | 13 | 51 | 1 week | | |
| | | | 7* | M | 5 | 126 | 2 months | | |
| | | | 8* | M | 3 | 146 | 1 week | | |
| | Progression-to-chronic ITP | n = 7 | 9* | M | 1 | 24 | 2 weeks | MG | |
| | (Blood was collected within 6 | | 10* | F | 1 | 6 | 2 months | steroids | |
| | months of diagnosis and the | | 11* | M | 1 | 39 | 2 months | | |
| | patient did not resolve by 6 | | 12* | F | 0 | 3 | 4 months | | |
| | months) | | 13 | M | 1 | 30 | 5 months | | |
| | | | 14 | F | 18 | 49 | 5 months | | |
| | | | 15 | F | 6 | 1 | 1 month | | |
| | Resolved acute ITP | n = 6 | 16 | M | 4 | 325 | 3 months | | |
| | (Blood was collected after the | | 17 | M | 5 | 249 | 6 months | | |
| | patient resolved from self-limited | | 18 | M | 1 | 342 | 8 months | | |
| | acute ITP) | | 19 | M | 1 | 210 | 3 weeks | MG | |
| | | | 20 | M | 4 | 270 | 2 months | | |
| | | | 21 | M | 4 | 270 | 1 month | | |
| | Chronic ITP | n = 15 | 22* | F | 7 | 8 | 2 years | | Resistant to multiple |
| | (Blood was collected after 6 | | | | | | | | treatments |
| | months of diagnosis when the | | 23* | F | 13 | 137 | 7 years | | |
| | patient had active disease) | | 24* | F | 18 | 3 | 6 years | Imuran | Resistant to multiple |
| | | | | | | | | | treatments, |
| | | | | | | | | | splenoctomized |
| | | | 25* | F | 8 | 111 | 1 year | | |
| | | | 26* | M | 5 | 19 | 8 months | steroids | |
| | | | 27* | F | 9 | 80 | 3 years | | |
| | | | 28* | M | 12 | 21 | 1 year | | |
| | | | 29* | M | 13 | 80 | 3 years | | |
| | | | 30* | M | 10 | 18 | 7 years | Imuran | Resistant to multiple |
| | | | | | | | | | treatments, |
| | | | | | | | | | splenoctomized |
| | | | 31* | M | 5 | 53 | 1 year | Exaction for | |
| | | | | | | | | [symbol] | |
| | | | 32* | M | 14 | 61 | 11 years | | Resistant to multiple |
| | | | | | | | | | treatments |
| | | | 33* | F | 5 | 85 | 3 years | | |
| | | | 34* | M | 18 | 19 | 9 months | steroids, MG | Resistant to multiple |
| | | | | | | | | | treatments, |
| | | | | | | | | | splenoctomized |
| | | | 35 | M | 11 | 9 | 8 years | Imuran, | Resistant to multiple |
| | | | | | | | | Prednisone | treatments; |
| | | | | | | | | | splenoctomized |
| | | | 36* | F | 13 | 59 | 9 months | | |
| Glutathione | Self-limited acute ITP | n = 5 | 37 | M | 4 | 105 | 1 month | | |
| measurement | (Blood was collected within 6 | | 38 | F | 5.5 | 126 | 2 months | | |
| by LC-MS/MS | months of diagnosis when the | | 39 | F | 13 | 128 | 5 months | | |
| | patients had active disease; | | 40 | M | 5 | 62 | 2 weeks | Steroids | |
| | patients resolved within 6 | | 41 | M | 1 | 128 | 5 months | Steroids | |
| | months) | | | | | | | | |
| | Progression-to-chronic ITP | n = 4 | 42 | F | 3 | 95 | 2 months | | |
| | (Blood was collected within 6 | | 43 | F | 4 | 4 | 2 months | | |
| | months of diagnosis and the | | 44 | M | 17 | 13 | 1 day | MG | |
| | patient did not resolve by 6 | | 45 | M | 13 | 47 | 6 months | Steroids | |
| | months) | | | | | | | | |
| | Outcome status pending | n = 2 | 46 | F | 4.5 | 4 | 2 days | | |
| | (Blood was collected within 6 | | 47 | M | 9 | 7 | 2 weeks | | |
| | months of diagnosis and whether | | | | | | | | |
| | the patient will resolve by 6 | | | | | | | | |
| | months remains pending) | | | | | | | | |
| | Chronic ITP | n = 16 | 48 | M | 5 | 4 | 2 years | | |
| | (Blood Was collected after 6 | | 49 | F | 9 | 4 | 2 years | | |
| | months of diagnosis when the | | 50 | M | 3 | 69 | 2 years | | |
| | patient had active disease) | | 51 | M | 12 | 80 | 2 years | | |
| | | | 52 | F | 17.5 | 94 | 6 months | | |
| | | | 53 | M | 16 | 21 | 14 years | | |
| | | | 54 | M | 7 | 41 | 2 years | | |
| | | | 55 | F | 16 | 88 | 1 year | | |
| | | | 56 | M | 14 | 27 | 11 years | | |
| | | | 57 | F | 8 | 4 | 1 year | | |
| | | | 58 | F | 11 | 18 | 5 years | | |
| | | | 59 | M | 4 | 36 | 1 year | | |
| | | | 60 | F | 5.5 | 121 | 1 year | | |
| | | | 61 | F | 4 | 132 | 3 years | | |
| | | | 62 | M | 16 | 34 | 2 years | Steroids | |
| | | | 63 | F | 6 | 131 | 2 years | Steroids | |

The samples marked with * symbol were used in microarray analysis Except for samples #1, 2, 6, 23 and 35, all the other samples numbered between 1 and 36 were used in realtime PCR analysis. [symbol]
indicates data missing or illegible when filed

### Microarray Procedure.

Total RNA was isolated from whole blood using the PAXgene™ Blood RNA System (PreAnalytiX, Hombrechtikon, Switzerland). The RNA from ITP patients and controls, along with Human Universal Reference RNA (Stratagene, La Jolla, Calif.), were linearly amplified using the MessageAmp™ aRNA amplification kit (Ambion, Austin, Tex.). Samples were labeled and hybridized to human cDNA microarrays (Stanford Functional Genomics Facility, Stanford, Calif.) using a previously published protocol. (Sood R et al., Proc Natl Acad Sci USA. 2006; 103(14):5478-83). The cDNA microarrays contained 41,805 spots corresponding to 24,473 unique putative genes. Image analysis was performed with Axon GenePix Pro® (Molecular Devices, Sunnyvale, Calif.). The data were then submitted to the Stanford Microarray Database, Stanford, Calif. for further analysis (http(colon)//smd(dot)stanford(dot)edu). The microarray data of this study have been deposited in NCBI's Gene Expression Omnibus (Edgar et al., 2002) and are accessible through GEO Series accession number GSE23754 (http(colon)//www(dot)ncbi(dot)nlm(doOnih(dot)gov/geo/query/acc.cgi?acc=GSE23754).

### Microarray Data Analysis

The microarray data were retrieved from the Stanford Microarray Database. The following criteria were used for selecting array elements with good quality: regression correlation >0.6; median fluorescent hybridization signal intensity divided by median background intensity >1.5 in both the sample and reference channels for at least 80% of the samples analyzed. Two-class unpaired SAM was performed to identify genes which were differentially expressed in chronic ITP compared to self-limited acute ITP. A d-score was assigned to each gene on the basis of change in gene expression relative to the standard deviation (SD) of repeated measurements. Permutations of the repeated measurements estimated the q-value, which is a false discovery rate-based measure of significance. (Tusher V G, et al., Proc Natl Acad Sci USA 2001; 98:5116-5121 and Eisen M B, et al., Proc Natl Acad Sci USA 1998; 95:14863-14868) To generate unsupervised clustering images using differentially expressed genes, the gene expression results of self-limited acute ITP patients, chronic ITP patients and normal controls were retrieved by IMAGE clone ID (including only putative genes) at a q-value of 0. The filters for result sets were set up as: ‘Spot is not flagged by experimenter,’ ‘Regression Correlation >0.6,’ ‘Ch1 Mean Intensity/Median Background Intensity >2.5,’ ‘Ch2 Normalized (Mean Intensity/Median Background Intensity)>2.5,’ ‘Genes were centered by mean,’ and ‘Only using genes with >80% good data’. Genes and arrays were both clustered by the Pearson Correlation. Unsupervised clustering images were created with the Gene Tree View program.

### Pathway Analysis.

Pathway analysis of transcripts with elevated expression in chronic ITP was performed using Ingenuity® Pathways Analysis (IPA® version 8.5, Redwood City, Calif., www(dot)ingenuity(dot)com) and aberrant functional networks and canonical pathways were recognized. IPA transforms a list of genes into a set of relevant networks based on the extensive records maintained in the Ingenuity® Pathways Knowledge Base (IPKB). IPA also performs statistical computing to identify the most significant ontologies, networks and canonical pathways based on the given list. The p-value associated with a function or a pathway is a measure of the likelihood that the association between a set of focus genes in the experiment and a given process or pathway is due to random chance; in general, a p-value (calculated using the right-tailed Fisher Exact Test) less than 0.05 indicates a statistically significant, non-random association. In this analysis, the Ingenuity® Pathways Knowledge Base (genes+endogenous chemicals) was chosen as the reference set. Both direct and indirect relationships were included and only molecules of human species were considered.

### Quantitative Real-Time PCR Validation.

Unamplified RNA samples isolated from whole blood were reverse transcribed to cDNA with High Capacity cDNA Reverse Transcription Kit (Applied Biosystems, Foster City, Calif.). The 25 μL reaction volume contained 12.5 μL TaqMan® Gene Expression Master Mix, 1.25 μL Taqman® gene expression assays, 5 μL cDNA sample, and 6.254 RNasefree water. All real-time PCR reagents were purchased from Applied Biosystems, Foster City, Calif. Real-time PCR was performed on a 7900HT Realtime PCR System with MicroAmp® Optical 96-Well Reaction Plate (Applied Biosystems, Foster City, Calif.). The relative quantification method was used per the manufacturer's instructions (www(dot)appliedbiosystems(dot)com, document number 040980) and standards were prepared from human bone marrow, brain or testis RNA (Clontech Laboratories, Mountain View, Calif.). Detailed information on Taqman® assays and the corresponding standards used in each experiment are listed in Table 2. Positive controls using Human Universal Reference RNA (Stratagene, Santa Clara, Calif.) and no-template controls were set up with each plate. Samples were run in triplicate and then normalized to the housekeeping gene GAPDH.

TABLE 2
Real-time PCR Taqman assays, standards and results.

| Gene | ABI Taqman | cDNA standard | | |
|---|---|---|---|---|
| symbol | assay ID | (human) | Analysis | p value |
| VNN1 | Hs01545812_m1 | Bone marrow | A vs A-C | 0.0093 |
| | | | N vs A-C | 0.0177 |
| | | | A vs N | 0.7242 |
| | | | A-R vs A-C | 0.0221 |
| | | | A vs RC | 0.0127 |
| AVIL | Hs00198114_m1 | Brain | A vs A-C | 0.0105 |
| | | | A vs N | 0.0295 |
| | | | N vs A-C | 0.0087 |
| RAPGEF2 | Hs00248010_m1 | Testes | A vs C | 0.0140 |
| NCOA1 | Hs00186661_m1 | Testes | A vs C | 0.0234 |
| SORL1 | Hs00268342_m1 | Testes | A vs C | 0.0234 |
| ACOX1 | Hs01074241_m1 | Testes | A vs C | 0.0280 |
| GNAQ | Hs00387073_m1 | Brain | A vs C | 0.0385 |
| DDEF1 | Hs00987469_m1 | Brain | A vs C | 0.0301 |
| GAPDH | | Bone marrow, | Endogenous | |
| | | Brain, or Testes | control | |

A: self-limited acute ITP; A-C: progression-to-chronic ITP: N: healthy controls; A-R: resolved acute ITP: RC: chronic ITP resistant to multiple treatments; C: chronic ITP

For the validation of differentially expressed genes between self-limited acute ITP and chronic ITP patients, pre-developed Taqman® assays targeting 6 genes—RAPGEF2, NCOA1, SORL1, ACOX1, GNAQ, and DDEF1—were performed in 18 samples for which sufficient amounts of RNA were available (5 self-limited acute ITP and 13 chronic ITP samples). For the validation of differentially expressed genes between self-limited acute ITP and progression-to-chronic ITP patients, pre-developed Taqman® assays targeting VNN1 and AVIL (Advillin) were used in 8 self-limited acute ITP and 7 progression-tochronic ITP samples. VNN1 expression was also validated with the same Taqman® assay in 8 self-limited acute ITP and 6 treatment-resistant chronic ITP patients.

### Expression Distribution of VNN1 in Subsets of Human Blood Cells.

CD15+ granulocytes were sorted from 2 blood donors by magnetic-activated cell sorting (MACS) with CD15 microbeads (Miltenyi Biotec Inc., Auburn, Calif.). CD20+ B cells, CD14+ monocytes, CD3+ CD4+ T cells, and CD3+ CD8+ T cells were sorted from 3 blood donors by fluorescence-activated cell sorting (FACS). Platelets (>99.9% pure) from 10 blood donors were obtained from the Stanford Blood Center. Total RNA was isolated from each subset of blood cells with Qiagen RNeasy® Mini Kit (QIAGEN, Valencia, Calif.) and reverse transcribed to cDNA with the High Capacity cDNA Reverse Transcription Kit (Applied Biosystems, Foster City, Calif.). Real-time quantitative PCR was performed as described above using pre-developed VNN1 and GAPDH Taqman® assays (Applied Biosystems, Foster City, Calif.), followed by normalization of VNN1 expression values to GAPDH as described above.

Treating Human PBMCs with Oxidative Stress Inducers In Vitro.

Buffy coats from healthy blood donors were obtained from the Stanford Blood Center. PBMCs were isolated by Ficoll-Paque™ PLUS gradient centrifugation (GE Healthcare, Pittsburgh, Pa.). Five samples were treated with sodium arsenite (Sigma-Aldrich, St. Louis, Mo.) at the final concentration of 5 μM and Lipopolysaccharide (LPS, Sigma-Aldrich, St. Louis, Mo.) at the final concentration of 10 ng/ml. The treated cells and the non-treated control cells were harvested 12 hours after treatment, then total RNA was extracted with the Qiagen RNeasy® Mini Kit (QIAGEN, Valencia, Calif.) and reverse transcribed to cDNA with the High Capacity cDNA Reverse Transcription Kit (Applied Biosystems, Foster City, Calif.). Real-time quantitative PCR was performed as described above using pre-developed VNN1, PPARγ and GAPDH Taqman assays (Applied Biosystems, Foster City, Calif.); VNN1 and PPARγ expression values were normalized to GAPDH.

### Measuring Glutathione Level in ITP Patient.

The levels of glutathione (GSH, reduced form) and glutathione disulfide (GSSG, oxidized form) in the whole blood of each subject were determined using a liquid chromatography-tandem mass spectrometry (LC-MS/MS)-based procedure modified from that of Norris et al. (J Chromatogr B Biomed Sci Appl. 2001; 762 (I):17-23) In brief, whole blood was mixed in a 1:4 (v:v) ratio with solution containing n-ethylmaleimide (NEM), sulfosalicylic acid, EDTA and methanol. Supernatants were diluted with stable-isotope internal standards (GSH-¹³C, ¹⁵N-NEM and GSSG-¹³C, ¹⁵N) and analyzed by LC-MS/MS, using an API 3000 Tandem Mass Spectrometer with Turbulon Ion Spray source, Shimadzu solvent delivery system and LEAP Technology autosampler. GSH (as GSH-NEM) and GSSG were monitored by transitions m/z 433→304 and m/z 613→3355 respectively, and data was acquired using Analyst 1.4.

### Statistical Analysis.

Significance analysis of microarrays (SAM) was conducted for analysis of microarray data to identify differentially expressed genes. The Mann-Whitney test was used in the analysis of real-time PCR validation data. Unpaired t-test was conducted for the comparison of glutathione levels in ITP patients and controls. Paired t-test was carried out for the analysis of differences in GSH/GSSG ratios in cells with or without LPS or sodium arsenite treatments.

### Results

### Identification of Differentially Expressed Genes Between Acute and Chronic ITP.

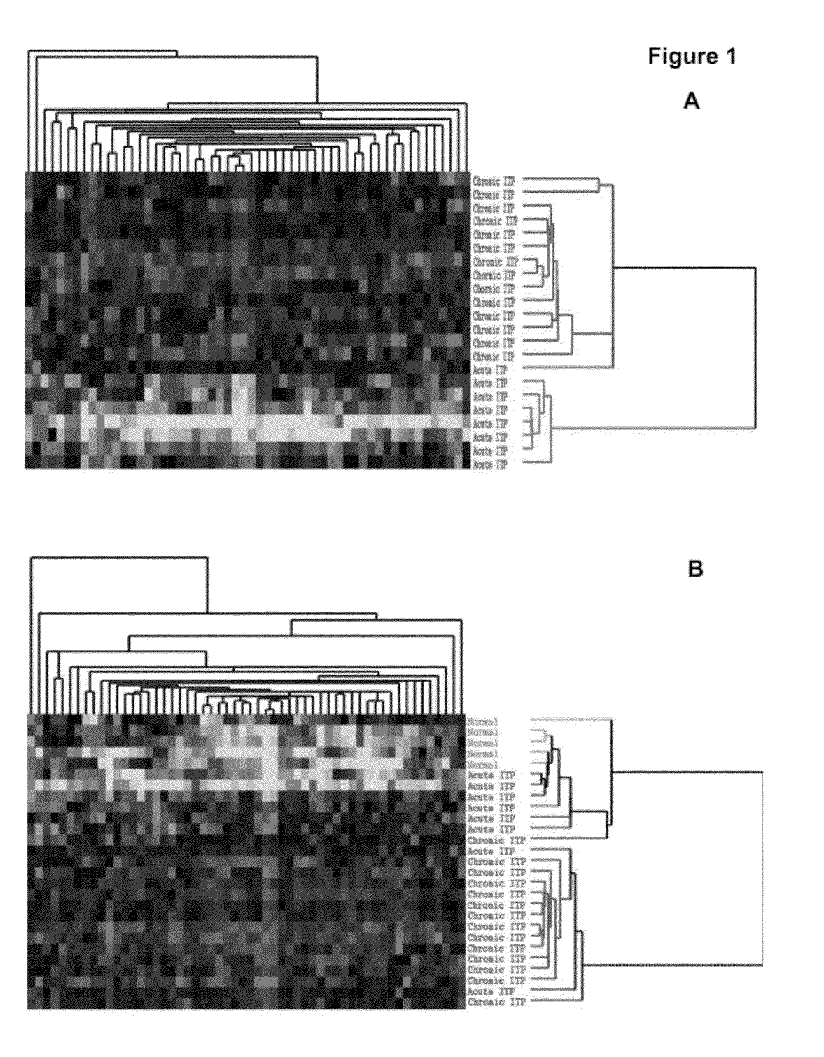

Whole blood from patients with acute and chronic ITP was subjected to cDNA microarray analysis. At a q-value of less than 5%, 535 transcripts were revealed to be over-expressed and 2 transcripts were under-expressed in chronic ITP (data not shown). At the highest significance level of q-value at 0, 69 transcripts were overexpressed in chronic ITP; screened with the filter setting described in the Material and Methods section, 57 biosequence IDs remained for generating unsupervised clustering image files. The clustering results are shown as heat maps in FIG. 1. FIG. 1A shows the hierarchical clustering of self-limited acute ITP and chronic ITP: the selected transcripts separate into two distinct subgroups. To learn whether either expression pattern is similar to that of healthy individuals, we added a healthy control group and used the same set of transcripts for unsupervised clustering. As shown in FIG. 1B, two expression clusters were revealed. The cluster on the left includes all of the healthy controls and the majority of self-limited acute ITP patients, while the cluster on the right contains mostly chronic ITP patients. The expression levels of these transcripts are lowest in normal controls, highest in chronic ITP patients, and slightly elevated in selflimited acute ITP patients.

Functional Network and Canonical Pathway Analysis of Over-Expressed Genes Associated with Chronic ITP.

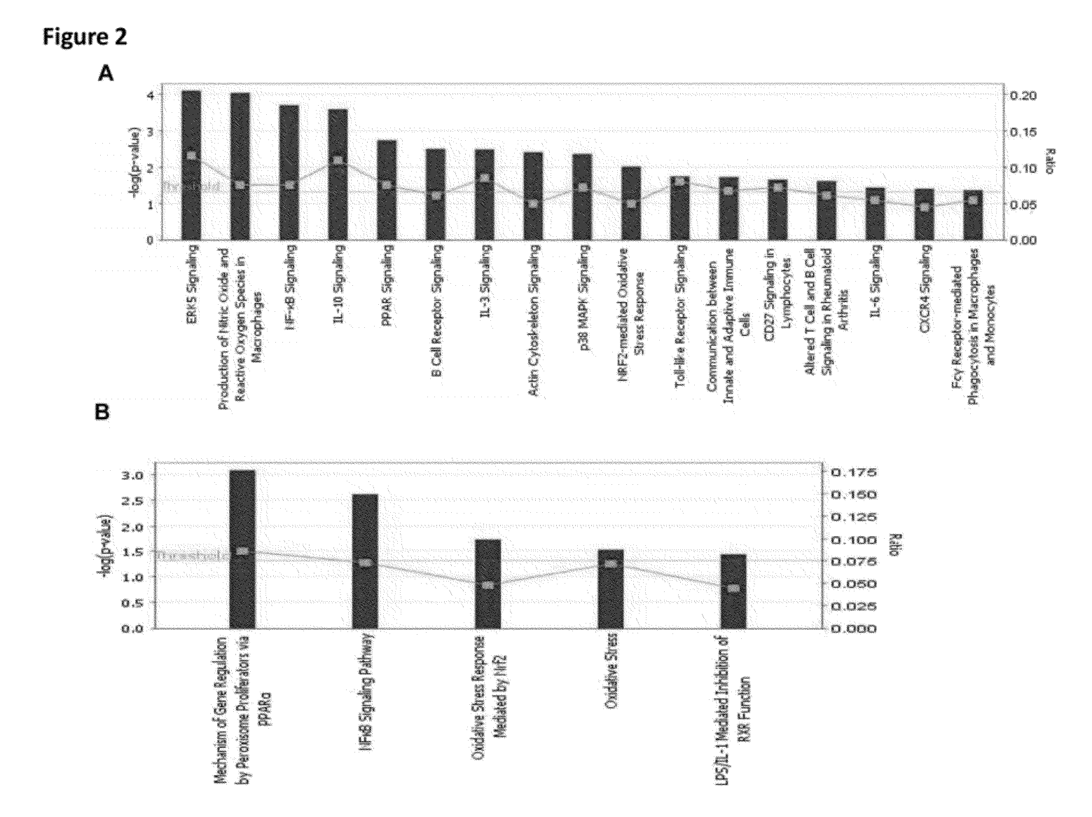

The over-expressed genes (q<5%) associated with chronic ITP were analyzed using Ingenuity® Pathway Analysis (IPA® version 8.5, Ingenuity Systems, Redwood City, Calif., http(colon)//www(dot)ingenuity(dot)com). The bar chart in FIG. 2A shows the statistically significant canonical pathways with biological relevance, including two direct oxidative stressrelated pathways—‘Production of Nitric Oxide and Reactive Oxygen Species in Macrophages’ and ‘NRF2-mediated Oxidative Stress Response.’ Other signaling pathways with high statistical significance include ERK5 (Extracellular signal-regulated kinase 5), NFκB (Nuclear factor kappa-light-chain-enhancer of activated B cells), IL-10 (Interleukin-10) and PPAR (Peroxisome proliferator-activated receptors). The pathway categories and focus genes of the significant canonical pathways are listed in Table 3. The over-expressed genes participating in the 2 oxidative stress-related pathways as well as 4 other highly significant canonical pathways were used to create the pathway graph shown in supplementary FIG. 1; the connections between the molecules are represented by lines. Toxicity lists are lists of molecules known to be involved in a particular type of toxicity, and IPA® scored the dataset against the known lists. As shown in FIG. 2B among the 5 toxicity lists that were significantly associated with chronic ITP, 2 were oxidative stress related sets, in addition to PPAR and NFκB signaling pathways.

TABLE 3
Canonical pathways and the corresponding over-expressed genes correlated with chronic ITP.

| | Pathway Categories | | | | | | | | |
|---|---|---|---|---|---|---|---|---|---|
| | Intracellular | | | | | | | | |
| | and | | | | | | | Cellular | |
| Ingenuity | second | Cellular | | Humoral | Nuclear | Organismal | | stress | |
| Canonical | messenger | immune | Cytokine | immune | receptor | growth and | | and | Genes overexpressed |
| Pathways | signaling | response | signaling | response | signaling | development | Apoptosis | injury | in chronic ITP |
| ERK5 | X | | | | | | | | GNAQ, RPSEKA5, FO5, |
| Signaling | | | | | | | | | MAP3K2, CREB5, MAP3K3, |
| | | | | | | | | | FOXO3, SGK1 |
| Production | | X | | | | | | | CREBBP, TLR2, SIRPA, |
| of | | | | | | | | | PPP1R3D, RAP1A, CYBS, |
| Nitric Oxide | | | | | | | | | FOS, PIK3CD, MAP3K2, |
| and | | | | | | | | | IFNG, NCF4, MAPJK3 |
| Reactive | | | | | | | | | |
| Oxygen | | | | | | | | | |
| Species in | | | | | | | | | |
| Macrophages | | | | | | | | | |
| NF-κB | | X | X | X | | | | | CREBBP, TLR2, GSK38, |
| Signaling | | | | | | | | | PIK3CD, TLR1, IL1R1, |
| | | | | | | | | | MAP4K4, MAP3K3, IL1R11, |
| | | | | | | | | | TNFSF138, IL1R2 |
| IL-10 | | X | X | | | | | | SOC53, FOS, IL1R1, MAP4K4, |
| Signaling | | | | | | | | | CCR1, IL1RN, IL1R2 |
| PPAR | | | | | X | | | | CREB3P, FOS, IL1R1, NCOA1, |
| Signaling | | | | | | | | | MAP4K4, IL1RN, IL1R2 |
| B cell | | | | X | | | | | LYN GSK36, PIK3CD, |
| Receptor | | | | | | | | | MAP3K2, GAB2, CREE5, |
| Signaling | | | | | | | | | MAP3K3 BCL6, PPP3CA |
| IL-3 | | X | | | | | | | FOS, CSF2RB, PK3CG, GAB2, |
| Signaling | | | | | | | | | PPP3CA, PAK1 |
| Actin | | | | | | X | | | MSN, PIK3CD, SSH2, |
| Cytoskeleton | | | | | | | | | IDGAP1, LIMK2, MYH9, PXN, |
| Signaling | | | | | | | | | IOGAP2, GSN, PPP1R12B, |
| | | | | | | | | | PAK1 |
| ERK/MAPK | X | | | | | | | | RPS6KA5, PPP1R3D, RAP1A, |
| Signaling | | | | | | | | | DUSP1, FOS, FIK3CD, |
| | | | | | | | | | CREB5, PXN, H3F3A, PAK1 |
| p38 MAPK | X | X | X | X | | | | X | RPS6KA6, OUSP1, IL1R1, |
| Signaling | | | | | | | | | CRE65, IL1R11, H3F3A, |
| | | | | | | | | | IL1R2 |
| NRF2- | | | | | | | | X | CRE85P, FOS, MAFG, GSK3B, |
| mediated | | | | | | | | | SOD2, PIK3CD, SOSTM1, |
| Signaling | | | | | | | | | GSTO2, GSTM3 |
| Stress | | | | | | | | | |
| Response | | | | | | | | | |
| Toll-like | | X | | X | | | X | | TLR2, FOS, TLR1, |
| Receptor | | | | | | | | | MAP4K4 |
| Signaling | | | | | | | | | |
| Communication | | X | | | | | | | TLR2, TLR1, IFNG, IL1RN, |
| between Innole | | | | | | | | | TNFSF13B |
| and adaptive | | | | | | | | | |
| immune | | | | | | | | | |
| Cells | | | | | | | | | |
| CD27 | | X | | | | | X | | FOS, MAP3K2, MAP3K3, BID |
| Signaling in | | | | | | | | | |
| Lymphocytes | | | | | | | | | |
| Altered | | X | | | | | | | TLR2, TLR1, IFNG, IL1RN, |
| T Cell and | | | | | | | | | TNFSF13B |
| B Cell | | | | | | | | | |
| Signaling in | | | | | | | | | |
| Rheumatoid | | | | | | | | | |
| Arthritis | | | | | | | | | |
| IL-6 Signaling | | X | X | | | | | | FOS, IL1R1, MAP4K4, IL1RN, |
| | | | | | | | | | IL1R2 |
| CXCR4 | | X | X | | | | | | GNAQ GNG10, LYN, FOS, |
| Signaling | | | | | | | | | PIK3C0, PXN, PAK1 |
| Fcγ | | X | | | | | | | LYN GAB2, PXN, CBL, PAK1 |
| Receptor- | | | | | | | | | |
| mediated | | | | | | | | | |
| Phagocytosis in | | | | | | | | | |
| Macrophages | | | | | | | | | |
| and Monocytes | | | | | | | | | |

Association of VNN1 Over-Expression with Disease Progression During the Acute Stage of ITP and Treatment Resistance in Chronic ITP

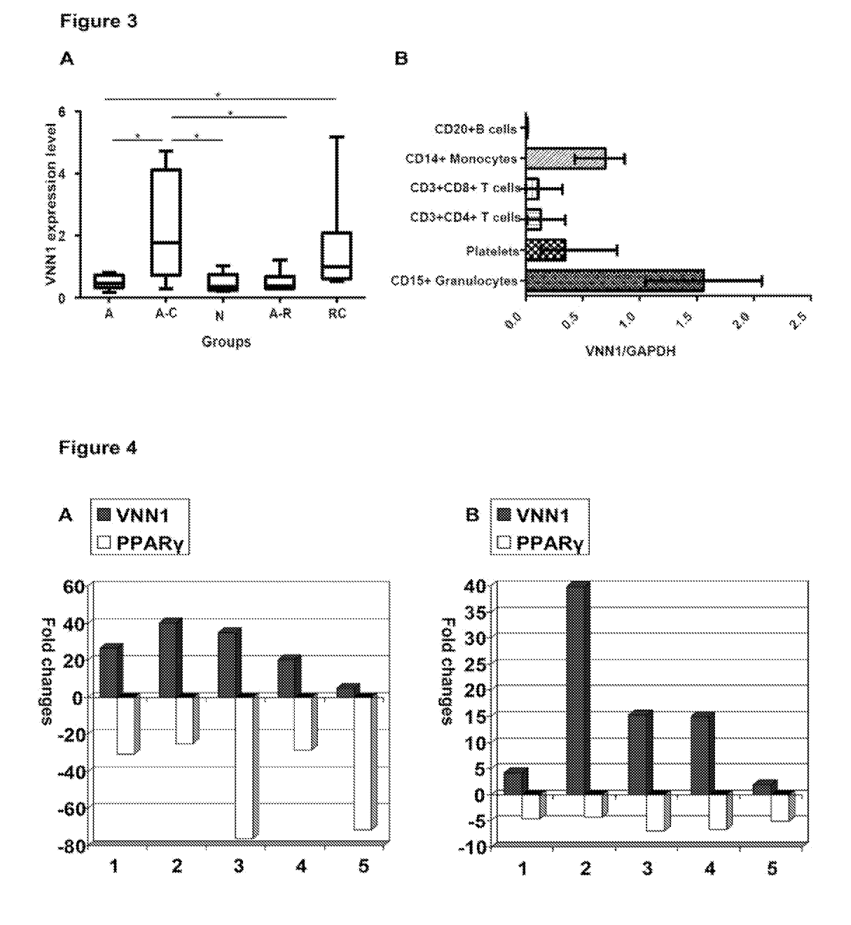

When we used two-class unpaired SAM to analyze the expression profiles of self-limited acute ITP patients and acute ITP patients who later progressed to chronic disease, 2 overexpressed genes were revealed at the q-value of 0: VNN1 (up-regulated 3.88 fold) and AVIL (up-regulated 2.15 fold). When SAM was applied to the expression profiles of self-limited acute ITP and treatment-resistant chronic ITP samples, VNN1 expression was increased in the latter group with a q-value of less than 5%. Based on this dataset, VNN1 is the only gene which was detected to be over-expressed in both the progression to chronic ITP patients and in treatment-resistant chronic ITP patients. Next, we used quantitative real-time PCR to measure VNN1 expression in the original 3 patient groups (self-limited acute ITP, progression-to-chronic ITP, and treatment-resistant chronic ITP) as well as 2 additional groups (healthy controls and resolved acute ITP). The results, presented in FIG. 3A, show that the increased expression of VNN1 in the progression-to-chronic ITP group (p=0.0093) and treatment-resistant chronic ITP group (p=0.0127) was validated by real-time PCR. Interestingly, VNN1 expression in the progression-to-chronic ITP group was also significantly higher than the normal control or resolved acute ITP groups, while the VNN1 expression of the latter two groups were comparable to that of the self-limited acute ITP group.

### Expression of VNN1 in Peripheral Blood Cells and Increased Expression in Response to Oxidative Stress Inducers

Since little is known about the expression and function of VNN1 in human blood cells, we examined its expression at the transcription level in platelets and the major white blood cell subsets. As shown in FIG. 3B, the relative expression level of VNN1 is high in CD15+ granulocytes and CD14+ monocytes, moderate in platelets, and low in CD3+ CD8+ T cells, CD3+ CD4+ T cells and CD20+ B cells. We subsequently asked whether VNN1 over-expression also correlates with oxidative stress in human blood cells. Low doses of lipopolysaccharide (LPS, 10 ng/ml) and sodium arsenite (5 μM), which are both oxidative stress inducers, were used to treat human PBMCs. The expression fold changes of VNN1 and PPARγ in treated cells compared to non-treated cells after 12 hours of treatment are shown in FIG. 4. In LPS treated cells, VNN1 expression increased 5.1˜40.2 fold while PPARγ expression decreased 24.8˜71.6 fold; in sodium arsenite treated cells, VNN1 expression increased 1.9˜39.8 fold while PPARγ expression decreased 4.3˜6.9 fold. To ensure that oxidative stress was indeed present after treatment, the glutathione (reduced form) to glutathione disulfide (oxidized form) ratio (GSH/GSSG), a parameter of cellular redox status, was measured by the highly sensitive and specific liquid chromatography-tandem mass spectrometry method in 4 other PBMC samples treated in the same way. There was a statistically significant decrease of the GSH/GSSG ratio in cells treated with either LPS (p=0.04) or sodium arsenite (p=0.01) compared with untreated cells at 12 hours, indicating the presence of treatment-induced oxidative stress.

Real-Time PCR Validation of Genes Associated with Chronic ITP.

Real-time PCR validation of the expression of six genes (RAPGEF2, NCOA1, SORL1, ACOX1, GNAQ, DDEF1) in 18 specimens demonstrated statistically significant p-values in all cases, as shown in Table 2.

### Unbalanced Redox State in ITP Patients Compared to Control Subjects.

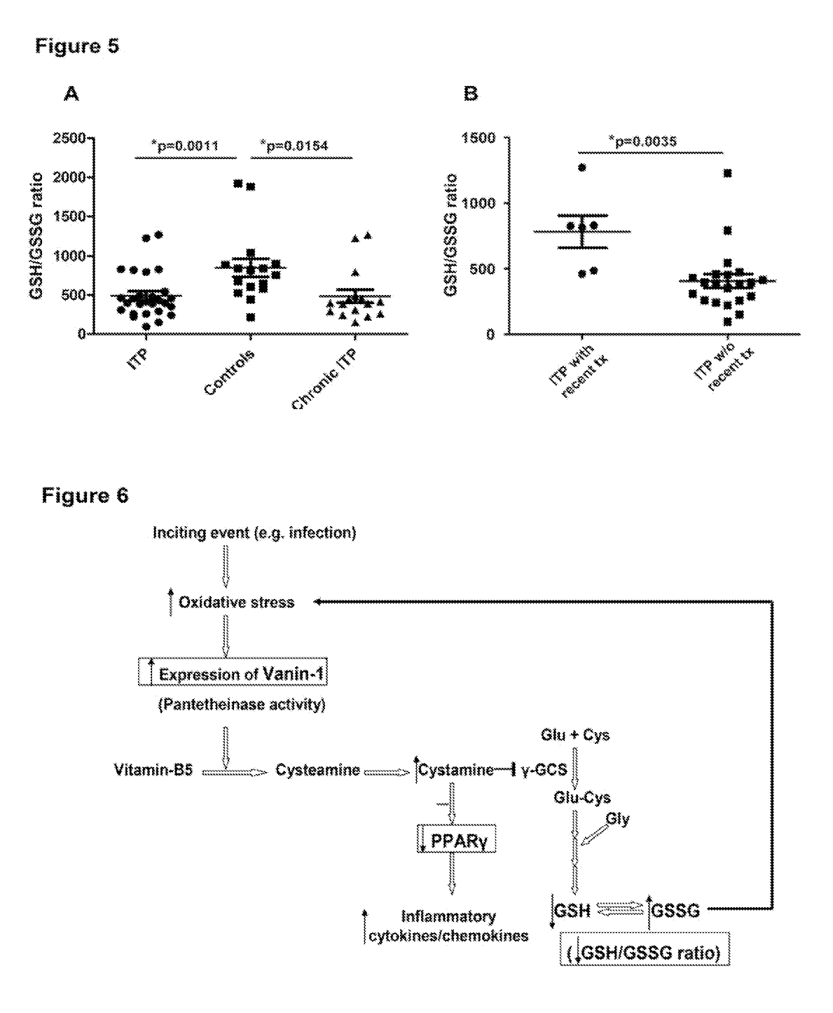

The ratio of reduced (GSH) to oxidized (GSSG) glutathione is an important parameter of redox status. GSH depletion, as indicated by a low GSH/GSSG ratio, is a hallmark of oxidative stress. The GSH/GSSG ratio was calculated for each sample. As shown in FIG. 5A, the whole blood GSH/GSSG ratio of the ITP group (both acute and chronic ITP patients) was significantly lower than that of the healthy controls (p=0.0011), indicating a higher oxidative stress state. The GSH/GSSG ratio in the chronic ITP group was also significantly lower than the control group (p=0.0154), while the difference between the self-limited acute ITP and control group did not reach statistical significance (p=0.0545). Another interesting finding, as shown in FIG. 5B, is that ITP patients with recent treatment (within one month of samples collection) had significantly higher GSH/GSSG ratio as compared to those without recent treatment. Thus, evidence of increased oxidative stress is exhibited in ITP patients in general and chronic ITP patients in particular; ITP patients with recent treatment (majority were treated with steroids) had ameliorated oxidative stress level.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of determining whether a subject is experiencing oxidative stress, said method comprising:
(a) evaluating the level of expression of a VNN1 expression product in cells of hematopoietic lineage (blood cells) from said subject to obtain a gene expression result; and
(b) determining whether said subject is experiencing oxidative stress based on said gene expression result, wherein an elevated level of expression of said VNN1 expression product in said blood cells indicates that said subject is experiencing oxidative stress.

2. The method of claim 1, wherein said blood cells are from a peripheral blood sample from said subject.

3. The method of claim 1, wherein said VNN1 expression product is a nucleic acid transcript.

4. The method of claim 3, wherein said evaluating step comprises performing one or more of: a PCR assay, an RT-PCR assay, a microarray assay, and a Northern blot.

5. The method of claim wherein said VNN1 expression product is a protein.

6. The method of claim 5, wherein said evaluating step comprises performing one or more of: flow cytometry, ELISA, immunohistochemistry, and Western blot analysis.

7. The method of claim 1, wherein the level of expression of one or more additional gene expression products is evaluated.

8. The method of claim 7, wherein the one or more additional gene expression products comprises a PPARγ expression product, wherein a reduced level of said PPARγ expression product in said blood cells indicates that said subject is experiencing oxidative stress.

9. The method of claim 1, wherein said determining step comprises comparing said gene expression result to a reference gene expression profile.

10. The method of claim 9, wherein said reference gene expression profile is selected from one or both of: an oxidative stress positive gene expression profile and a an oxidative stress negative gene expression profile.

11. A method of managing treatment in a subject having ITP, said method comprising:
(a) determining whether said subject having ITP is experiencing oxidative stress according to claim 1; and
(b) managing treatment of said subject having ITP based on said determining step (a).

12. A kit for determining whether a subject is experiencing oxidative stress, said system comprising:
(a) a gene expression evaluation element for evaluating the level of expression of a VNN1 expression product in blood cells from said subject to obtain a gene expression result; and
(b) an oxidative stress determination element for employing said gene expression result to determine whether said subject is experiencing oxidative stress.

13. The kit of claim 12, wherein said VNN1 expression product is selected from: a nucleic acid transcript and a protein.

14. The kit of claim 12, wherein said gene expression evaluation element comprises at least one reagent for assaying a blood sample for a VNN1 expression product.

15. The kit of claim 14, wherein said at least one reagent is selected from one or more of: an antibody, a nucleic acid probe, PCR primers, microarray, enzymes, buffers, control samples or reagents, and signal generating and detecting reagents.

16. The kit according to claim 14, wherein the gene expression evaluation element is configured for evaluating the level of expression of expression products for one or more additional genes, wherein said one or more additional genes comprises PPARγ.

17. The kit of claim 14, wherein said kit is configured for performing one or more of the following assays: PCR, RT-PCR, northern hybridization, microarray analysis, flow cytometry, ELISA, western blot, and immunohistochemistry.

18. A computer program product for determining whether a subject is experiencing oxidative stress, wherein said computer program product, when loaded onto a computer, is configured to employ a gene expression result from blood cells derived from said subject to determine whether said subject is experiencing oxidative stress, wherein said gene expression result comprises expression data for VNN1.
